(19) United States
(12) Patent Application Publication
(10) Pub. No.: US 2024/0254425 A1
(43) Pub. Date: Aug. 1, 2024

(54) MICROFLUIDIC PARTICLE SORTING

(71) Applicant: NODEXUS INC., San Jose, CA (US)

(72) Inventors: Hany Nassef, Hayward, CA (US); Anand Kesavaraju, Hayward, CA (US); Karthik Balakrishnan, Hayward, CA (US); Zhiliang Wan, Hayward, CA (US)

(21) Appl. No.: 18/415,313

(22) Filed: Jan. 17, 2024

Related U.S. Application Data
(60) Provisional application No. 63/439,733, filed on Jan. 18, 2023.

(51) Int. Cl. C12M 1/34; C12M 1/00; C12M 1/26; C12M 3/00; C12M 3/06

(52) U.S. Cl. CPC C12M 41/46; C12M 23/16; C12M 23/40; C12M 23/42; C12M 29/06; C12M 33/14

(57) ABSTRACT

Systems and methods for rapid detection and sorting of target particles based on specific characteristics are provided. Optical, electrical, or other detection of the target characteristic in a target particle in a microfluidic sample flow can be used to identify that target particle which can then trigger accurate downstream diversion and isolation of the target particle from the sample flow.

### CROSS-REFERENCE TO RELATED APPLICATIONS

This application claims priority to, and the benefit of, U.S. Provisional Application No. 63/439,733, filed Jan. 18, 2023, the content of which is incorporated by reference herein in its entirety.

### FIELD OF THE INVENTION

The invention relates to systems and method for identifying and isolating target particles from a sample.

### BACKGROUND

Existing live, single-cell isolation methods include fluorescence-activated cell sorting (FACS), magnetic-activated cell sorting (MACS), serial dilution, micromanipulation, and manual-picking. FACS and MACS are difficult to optimize and are often restricted to core facilities, and consecutive samples analyzed by the same fixed system pass through the same flow cells, potentially leading to sample contamination. Serial dilution involves diluting a sample containing cells of interest until there is a statistical likelihood that each aliquot of the diluted sample contains exactly one cell. This technique is labor intensive and results in a low isolation frequency. A high percentage of the target cells of interest may not be present in the input sample, and serial dilution does allow for any criteria for target selection for which cells are isolated. Additionally, serial dilution results in a high variability and lack of reproducibility from isolation to isolation. Micromanipulation and manual-picking involve the physical selection and isolation of individual cells, but these methods are labor intensive and low throughput.

### SUMMARY

The invention provides systems and methods for rapid detection and sorting of target particles based on specific characteristics. Optical, electrical, or other detection of the target characteristic in a target particle in a microfluidic sample flow can be used to identify that target particle which can then trigger accurate downstream diversion and isolation of the target particle from the sample flow. As opposed to many current methods wherein all target particles are diverted together into a collection tube, systems and methods of the current invention can be used to divert and isolate each target particle into a separate well for further use or analysis. Furthermore, the entire process may be automated, allowing for rapid particle sorting and isolation in a compact, integrated system with minimal user input required.

Systems and methods of the invention use a microfluidic chip to flow a sample fluid through a sensing region and subsequent sorting region and into a waste channel and reservoir. Upon sensing a target particle in the sensing region, the sorting region can divert that single particle out of the sample flow, through a branch channel, to a target channel for subsequent analysis or processing or, in preferred embodiments, for dispensing through a nozzle into an individual well. A plate or the chip may be manipulated via a computer-controlled stage such that each isolated target particle can be dispensed into its own well without disrupting sample flow. Target particles can be diverted through application of a trigger flow to the sorting region through a trigger channel positioned opposite an inlet to the branch channel. In order to accurately model flow characteristics and predict target particle position in the system, 3-D flow is undesirable and can be essentially prevented by limiting channel depth in the sorting and/or sensing regions. The channels may have greater depths outside of those regions but ramp or otherwise transition to the desired shallower depths as they approach or depart the sorting region.

In some embodiments, one or more of sample, trigger, carrier, and sheath fluids may be supplied via pressurized reservoirs onboard the cartridge or supplied with pressurized fluid via separate or combined inlets through an interface with an instrument. Similarly, waste fluid (e.g., sample fluid and non-target particles) may be collected in a waste reservoir positioned onboard the cartridge. In such embodiments, accumulated waste in the waste reservoir could serve to increase backpressure in the waste channel feeding the reservoir, thereby disrupting the flow characteristics of the device. Accordingly, in certain embodiments, smaller volume vertical towers may be incorporated to feed the waste reservoir by filling with a small volume of waste before spilling down into the waste reservoir. That way, the backpressure is limited to the force of the constant small volume in the tower and isolated from the fluctuating larger volume of spilled waste in the reservoir proper.

An additional problem caused by incorporating fluid reservoirs above the microfluidic chip is disruption of optical paths to the sensing region from fluid in those reservoirs. Accordingly, in certain embodiments, cartridges may include a viewport positioned in one or more of the reservoirs having at least one vertical wall to hold back fluid and provide an unobstructed optical path through the reservoir(s) to the sensing region below for target detection.

In some embodiments, trigger, sample, carrier, and/or sheath fluid sources may be independently driven or valved so that flow of each can be independently controlled. As such, it may be desirable to, upon detecting a target particle, both initiate a trigger flow to divert the target particle to the branch channel while also stopping flow of the sample in the sample channel. Stopping the sample flow during a sorting action can have benefits such as 1) slowing down flow such that flow and run time during non-sorting periods can be at a higher rate without requiring as high-speed valve actions and 2) allowing for trigger flow to continue to be driven while sample does not flow to the nozzle after a sorting action. This can accelerate dispensing time of the target particle by increasing the driving pressure of the trigger fluid and can also serve to pause target detection while the current particle is sorted. The system can thereby avoid being overwhelmed and potentially missing target particles in close proximity to one another in the sample flow. In some embodiments, a sorting action may include an initial diversion of a target particle into the branch channel via a short application of the trigger fluid. Subsequently, while the target particle is flowing through the branch and/or target channels, the sample flow may be stopped and the trigger flow may be again initiated in order to accelerate drop dispensing at the end of the target channel.

In certain embodiments, systems and methods of the invention may relate to alignment of the cartridge within the instrument. Precise alignment at the cartridge/instrument interface can be extremely important for proper target identification and sorting. Detection elements such as optical sensors are present in the instrument but must be precisely aligned with the sensing region and, where applicable, viewport in the cartridge in order to identify target particles in the microfluidic sample flow channel. Additionally, pressure manifolds for driving fluid flow from cartridge-based reservoirs and port interfaces for introducing instrument-based fluids into the microfluidic chip must be properly aligned to function. In certain embodiments, the cartridge and/or the instrument may comprise alignment features to avoid the need for motorized or manual positioning systems. Such features may include interacting raised or recessed elements configured to interact to precisely position the cartridge in a cradle in the instrument. Features may be asymmetric to only allow the cartridge to be placed in a single orientation. For example, the cartridge may comprise a skirt while the instrument cradle comprises a complimentary valley to receive the skirt. The skirt and valley may only partially extend around the perimeter of the cartridge (e.g., three sides of a square) such that a single orientation is permitted. The skirt and valley can also provide a rough alignment depending on the relative sizes of the two elements. In certain embodiments, a skirt and valley may be configured to provide a rough alignment while additional features provide final alignment. For example, kinematic coupling may be used. In certain embodiments one, two, or preferably three hemispherical features may be used on the mating surface of one of the instrument cradle or cartridge with corresponding v-grooves on the other mating surface to precisely align the cartridge in the cradle.

In some embodiments, the instrument may include a drop sensor operably associated with the nozzle of the cartridge when properly positioned in the instrument. The drop sensor may comprise, for example, reflective optical sensors with transistor outputs, slot type optical sensors, acoustic sensors, camera elements, pressure sensors, capacitive sensors, and in-line flow sensors. The drop sensor may detect the presence, size, and/or rate of formation of a meniscus at the nozzle or drop that has fallen out of the nozzle in order to predict when the next droplet will separate from the nozzle. This information can be used by the processor to predict which droplet will contain a target particle that has been diverted to the nozzle and therefore be used to inform nozzle placement over a waste or target receptacle for particle isolation and waste collection. This information can also be used to assess and calibrate flow through the cartridge.

Aspects of the invention can include a particle sorting cartridge comprising a sorting region. The sorting region can include a sample flow channel leading from a sensing region into a waste channel and having at least a first side and a second side substantially opposite the first side; a branch channel having an inlet on the first side of the sample flow channel and positioned at an angle acute to a direction of flow in the sample flow channel at the sorting region; and a trigger channel having an outlet on the second side of the sample flow channel. Each of the sample flow channel, waste channel, branch channel, and trigger channel may have a channel depth of about 200 μm or less within the sorting region. In certain embodiments, one or more of the sample flow channel, waste channel, branch channel, and trigger channel may have a channel depth greater than about 200 μm before or after the sorting region before transitioning to the shallower depth of the sorting region. In various embodiments, the shallow depth of one or more of the channels in the sorting region may be about 100 μm or less, 150 μm or less, about 200 μm or less, about 250 μm or less, about 300 μm or less, about 350 μm or less, about 400 μm or less, about 500 μm or less, about 600 μm or less, or about 700 μm or less, with one or more of the channels increasing to correspondingly greater depths outside of the sorting region

The branch channel may lead away from the sorting region to a target channel. The target channel can be operably associated with a nozzle. Particle sorting cartridges may further comprise a carrier channel connected to the target channel. The carrier channel and the branch channel can intersect at an inlet to the target channel. In certain embodiments, each of the waste channel, branch channel, and trigger channel may have a channel depth greater than about 200 μm before or after the sorting region while the sample channel has as channel depth of about 200 μm or less between the sensing region and the sorting region. In some embodiments, one or more of the sample flow channel, waste branch channel, and trigger channel may have a channel depth greater than about 200 μm before or after the sorting region and comprise a ramped region wherein channel depth transitions from greater than about 200 μm to about 200 μm or less. One or more of the channels may change width along with depth as they approach the sorting region. In some embodiments, one of more the channels may narrow in width as the depth transitions from greater than about 200 μm to about 200 μm or less. In certain embodiments the ramped region of the transition may have an average slope of between about 5 degrees and about 30 degrees. In some embodiments, the transition may have an average slope of about 5 degrees to about 50 degrees or about 50 degrees to about 90 degrees. In some embodiments, the transition may comprise a stepped region wherein channel depth transitions from greater than about 200 μm to about 200 μm or less. In certain embodiments, channel depths within the sorting region, as discussed above, may be about 150 μm or less or even about 100 μm or less while having greater channel depths outside of the sorting region in the manner discussed above.

Cartridges of the invention may include a branch channel inlet having an upstream edge substantially aligned with an upstream edge of the trigger channel outlet across the sample flow channel in the sorting region. Cartridge may further comprise a first sheath channel having an outlet on the first side of the sample flow channel at a point upstream of the sorting region and a second sheath channel having an outlet on the second side of the sample flow channel at the point upstream of the sorting region. The first and second sheath channels can intersect at an inlet fed by a single sheath fluid reservoir. The cartridge may further comprise a carrier channel connected to the target channel, wherein the carrier channel is fed by the single sheath fluid reservoir.

In certain aspects, methods of the invention may include methods for sorting a target particle from a sample including steps of: flowing a sample in a sample flow channel through a sensing region, through a sorting region, and into a waste channel, the sample flow channel having at least a first side and a second side substantially opposite the first side; and detecting a target particle in the sample at the sensing region and applying a trigger flow from a trigger channel having an outlet on the second side of the sample flow channel at the sorting region to divert the target particle from the sample flow channel into an inlet of a branch channel positioned on the first side of the sample flow channel at an angle acute to a direction of flow in the sample flow channel at the sorting region. Each of the sample flow channel, waste channel, branch channel, and trigger channel may have a channel depth of about 200 μm or less (or, in certain embodiments, 150 μm or less or 100 μm or less) within the sorting region and one or more of the sample flow channel, waste channel, branch channel, and trigger channel have a channel depth greater than about 100 μm before or after the sorting region. Methods may include flowing the target particle through the branch channel to a target channel.

In some embodiments, the target channel may be operably associated with a nozzle and the method may further comprise dispensing the target particle from the nozzle. Methods of the invention may include flowing a carrier fluid in a carrier channel connected to the target channel, the carrier channel and the branch channel intersecting at an inlet to the target channel. Methods may include maintaining a fluidic pressure in a carrier channel connected to the target channel and the branch channel to resist flow of the sample from the sample channel into the inlet of the branch channel in the absence of the trigger flow.

In some embodiments, methods may comprise flowing a sheath fluid in a first sheath channel having an outlet on the first side of the sample flow channel at a point upstream of the sorting region and a second sheath channel having an outlet on the second side of the sample flow channel at the point upstream of the sorting region. Methods of the invention may include flowing a carrier fluid in a carrier channel connected to the target channel, the carrier channel and the branch channel intersecting at an inlet to the target channel, wherein the carrier channel is fed by the single sheath fluid reservoir.

Aspects of the invention may include a particle sorting cartridge, comprising a microfluidic chip. The microfluidic chip may comprise a sample flow channel leading laterally from a sample reservoir positioned above the microfluidic chip, through a sensing region and into a sorting region; and a waste channel leading laterally from the sorting region to a waste reservoir inlet. The sorting cartridge can further include a waste reservoir positioned above the microfluidic chip and a viewport positioned vertically within one or more of the waste reservoir and the sample reservoir and directly above the sensing region, the viewport comprising at least one vertical wall operable to provide an optical path through one or more of the waste reservoir and the sample reservoir to the sensing region from above, the optical path being free from sample fluid and waste fluid.

Sorting cartridges of the invention can comprise a tower positioned vertically in the waste reservoir above the waste reservoir inlet, the tower having an outlet spaced vertically above a floor of the waste reservoir, the tower configured such that waste fluid from the microfluidic chip flows laterally through the waste channel into the waste reservoir inlet then vertically through the tower, then out of the outlet into the waste reservoir below the outlet, thereby limiting backpressure in the waste channel as waste fluid accumulates in the waste reservoir.

The waste reservoir may have a volume of about 30 milliliters, or between about 20 mL and 40 mL. The tower can have a volume of about 100 microliters (μL). The outlet may be positioned on the tower about 15-20 millimeters (mm) above the waste reservoir inlet. The waste reservoir can have a greater volume than the sample reservoir in order to ensure it does not overflow during sorting. In some embodiments, the sample reservoir may include an angled floor operable to direct fluid in the sample reservoir to an inlet to the sample flow channel positioned at a lowest point in the sample reservoir. The sample flow channel may include a filter between the inlet and the sensing region operable to allow target particles to pass therethrough but prevent passage of objects larger than a narrowest cross-sectional dimension in the sample flow channel without blocking flow through the filter. The filter can comprise a plurality of microposts. The sheath, carrier, and trigger flow channels can likewise have filters.

In certain embodiments, the sample flow channel can include a first side and a second side substantially opposite the first side, the microfluidic chip further comprising: a branch channel having an inlet on the first side of the sample flow channel; and a trigger channel having an outlet on the second side of the sample flow channel substantially aligned with the branch channel inlet. The particle sorting cartridge can further comprise a trigger channel inlet port operable to receive trigger fluid through a cartridge interface. The sample reservoir may be operable to interface with a pressure manifold through a cartridge interface.

In certain aspects, methods for sorting a target particle are described, the methods comprising: providing a particle sorting cartridge comprising a microfluidic chip. The microfluidic chip may include a sample flow channel leading laterally from a sample reservoir positioned above the microfluidic chip, through a sensing region and into a sorting region; and a waste channel leading laterally from the sorting region to a waste reservoir inlet. The particle sorting cartridge may further include a waste reservoir positioned above the microfluidic chip; and a viewport positioned vertically within one or more of the waste reservoir and the sample reservoir and directly above the sensing region, the viewport comprising at least one vertical wall operable to provide an optical path through one or more of the waste reservoir and the sample reservoir to the sensing region from above, the optical path being free from sample fluid and waste fluid. Methods can further include flowing a sample fluid from the sample reservoir through the sample flow channel, sensing region, sorting region, and waste channel into the waste reservoir; detecting a target particle through the viewport at the sensing region using an optical sensor; and diverting the target particle into a branch channel at the sorting region.

In certain embodiments, the particle sorting cartridge may further comprise a tower positioned vertically in the waste reservoir above the waste reservoir inlet, the tower having an outlet spaced vertically above a floor of the waste reservoir, and the method can include flowing waste fluid laterally through the waste channel into the waste reservoir inlet then vertically through the tower, then out of the outlet into the waste reservoir below the outlet, thereby limiting backpressure in the waste channel as waste fluid accumulates in the waste reservoir.

Methods may include directing sample fluid in the sample reservoir to an inlet to the sample flow channel positioned at the lowest point of the sample reservoir using an angled floor of the sample reservoir. In some embodiments, methods can comprise filtering objects larger than a narrow cross-sectional dimension in the sample flow channel from entering the sample flow channel using a filter between the inlet and the sensing region while allow target particles to pass therethrough without blocking flow through the filter.

The sample flow channel may comprise a first side and a second side substantially opposite the first side, the microfluidic chip further comprising: a branch channel having an inlet on the first side of the sample flow channel; and a trigger channel having an outlet on the second side of the sample flow channel substantially aligned with the branch channel inlet. Diverting the target particle into the branch reservoir can then include applying a trigger flow from the trigger channel upon detection of the target particle.

The particle sorting cartridge can further comprise a trigger channel inlet port and applying the trigger flow may include introducing fluid to the trigger channel via the trigger channel inlet port using a cartridge interface. Methods may include driving sample flow through the sample flow channel by applying pressure to the sample reservoir through a pressure manifold coupled to the sample reservoir through a cartridge interface.

Aspects of the invention may include methods for sorting a target particle from sample including flowing a sample in a sample flow channel through a sensing region, through a sorting region, and into a waste channel, the sample flow channel having at least a first side and a second side substantially opposite the first side; detecting a target particle in the sample at the sensing region; applying a trigger flow from a trigger channel having an outlet on the second side of the sample flow channel at the sorting region to divert the target particle from the sample flow channel into an inlet of a branch channel positioned on the first side of the sample flow channel; and stopping flow of the sample in the sample flow channel while or soon after the trigger flow is applied.

The branch channel inlet may be positioned at an acute angle relative to flowing sample in the sample flow channel in the sensing region. Methods may include flowing the target particle through the branch channel to a target channel through application of the trigger flow.

The target channel can be operably associated with a nozzle and the method may further comprise dispensing the target particle from the nozzle. Methods may comprise reinstituting flow of the sample in the sample flow channel after the target particle is dispensed. Flow may be reinstituted after the target particle enters the target channel. In some embodiments, flow may be reinstituted after the target particle is diverted to the branch channel. Further methods may include flowing a carrier fluid in a carrier channel connected to the target channel, wherein the carrier channel and the branch channel intersect at an inlet to the target channel.

In some embodiments, a fluidic pressure may be maintained in a carrier channel connected to the target channel and the branch channel to resist flow of the sample from the sample channel into the inlet of the branch channel in the absence of the trigger flow.

In some aspects of the invention, a particle sorting system is disclosed which may comprise a particle sorting cartridge in turn comprising: a sensing region operable to detect a target particle in a sample and a sorting region. The sorting region can include a sample flow channel leading from the sensing region into a waste channel and having at least a first side and a second side substantially opposite the first side; a branch channel having an inlet on the first side of the sample flow channel; and a trigger channel having an outlet on the second side of the sample flow channel. A sample fluid source can be operably associated with the sample flow channel, a trigger fluid source can be operably associated with the trigger channel, and a processor may be provided that is operable to receive sensing data from the sensing region and, upon detection of a target particle, apply a trigger flow from the trigger fluid source and stop a sample flow from the sample fluid source. One or more of the sample fluid source and the trigger fluid source can comprise a reservoir, a pressurized air supply, and valve controlled by the processor. One or more of the sample fluid source and the trigger fluid source can comprise a pump controlled by the processor. Systems may include a first sheath channel having an outlet on the first side of the sample flow channel at a point upstream of the sorting region and a second sheath channel having an outlet on the second side of the sample flow channel at the point upstream of the sorting region. The first and second sheath channels can intersect at an inlet fed by a single sheath fluid reservoir or two or more sheath fluid reservoirs, and the system may include a sheath fluid source operably associated with the first and second sheath channels, the processor being further operable to stop a sheath fluid flow upon detection of the target particle and application of the trigger flow.

### BRIEF DESCRIPTION OF THE DRAWINGS

FIG. 1 shows an exemplary microfluidic chip according to certain embodiments.

FIG. 2 shows a sorting region with partial switching or diversion of the sample fluid.

FIG. 3 shows a sorting region with incomplete switching or diversion of the sample fluid.

FIG. 4 shows a sorting region with complete switching or diversion of the sample fluid.

FIG. 5 illustrates a microfluidic chip comprising sheath channels.

FIG. 6 illustrates a microfluidic chip comprising sheath channels and lacking a carrier channel.

FIG. 7 shows a sorting region with ramped channel depths.

FIG. 8 illustrates target particle sorting in a microfluidic chip with a carrier waste channel.

FIG. 9 illustrates target particle sorting in a microfluidic chip without a carrier waste channel.

FIG. 10 shows a multi-axis motion stage for manipulating a collection plate or tubes relative to the dispensing nozzle.

FIG. 11 shows alignment features on an exemplary cartridge.

FIG. 12 shows alignment features on an exemplary cartridge interface.

FIG. 13 shows an exemplary drop sensor.

FIG. 14 shows particle dispensing estimation using a drop sensor.

FIG. 15 shows an exemplary cartridge and reservoirs according to certain embodiments.

FIG. 16 shows an exemplary cartridge having a viewport and waste reservoir towers.

FIG. 17 shows fluid level sensing using pressure feedback in cartridge reservoirs.

FIG. 18 illustrates ramped channels to reduce channel depth at the sorting region.

FIG. 19 illustrates an exemplary toroidal mirror useful to maintain optical accuracy when using moving parts such as when placed on a hinged lid.

FIG. 20 illustrates a dual parabolic mirror arrangement for optical particle detection according to certain embodiments.

### DETAILED DESCRIPTION

Systems and methods of the invention provide rapid, automated identification and sorting of individual particles of interest in a compact, user-friendly manner with disposable cartridges and minimal user input. Sorted particles may be individually isolated via automatic dispensing from a nozzle into separate wells or other vessels.

### Particle Sorting

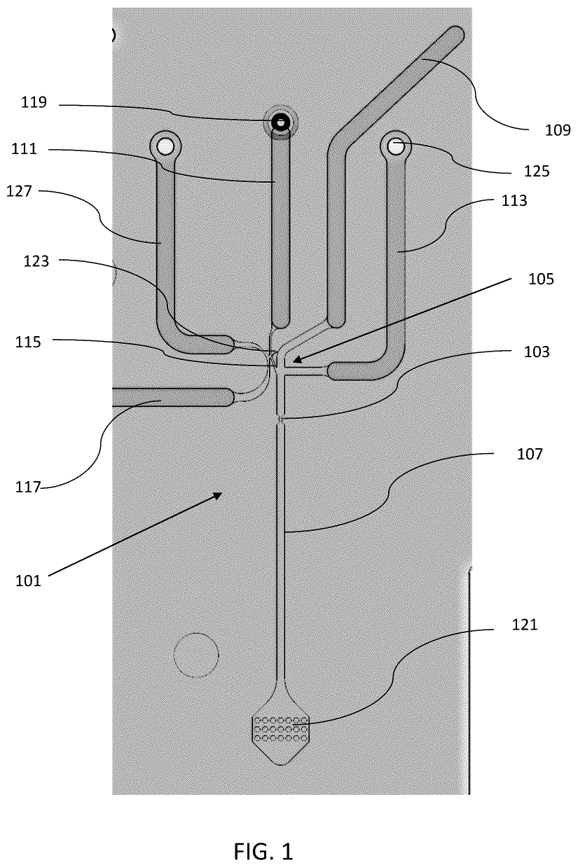

FIG. 1 illustrates an exemplary microfluidic chip 101 of a particle sorting cartridge according to certain embodiments. The primary elements include a sample flow channel 107 which can receive sample fluid from a sample reservoir positioned above the microfluidic chip 101 in the cartridge. The inlet to the sample flow channel 107 may include a filter 121 which may comprise a series of microposts operable to allow target particles (e.g., cells) to pass therethrough while blocking objects larger than the narrowest portion of the downstream channels (e.g., the sensing region) from entering the system and causing a catastrophic blockage. The array of microposts can allow sample flow to continue even if one or more large objects are caught between some of the microposts. Sample flows through the sample flow channel 107 through a sensing region 103. The sensing region 103 may have a channel width configured to encourage passage of a single particle therethrough at a time to ensure each particle can be individually assessed and, if desired, isolated. For example, the channel width in the sensing region 103 may be about the size of the sample particles being analyzed. Before or after the sensing region 103, channel width may be increased. Increasing the channel width after the sensing region may reduce the relative speed of the sample flow through the sorting region to provide processing time for identifying a target particle for sorting.

The sample flow channel 107 can continue through the sensing region 103 into a sorting region 105 wherein default flow will continue into a waste channel 109. Upon detection of a target particle in the sensing region 103, a trigger flow can be applied from an outlet of a trigger channel 113. The trigger channel may be connected to a trigger fluid source at a trigger channel inlet port 125. Pressurized trigger fluid, as with any fluid in the system, can be provided from an onboard reservoir in the cartridge or from an external source in the instrument. In the case of internal reservoirs, cartridges may interface with the instrument to seal the reservoir against a pressure manifold operable to provide pneumatic pressure or otherwise physically drive fluid from the reservoir into the associated channel. In some embodiments, external fluid sources may be housed on the instrument and can be delivered to the cartridge via pumps, valves, pneumatics, or other methods through an interface between the cartridge and the instrument. In preferred embodiments, the sample is housed in a sample reservoir on the cartridge and all other required fluids are housed on the instrument in bulk and provided to the cartridge as needed through an interface and inlet ports such as the trigger channel inlet port. Fluid sources may be pressurized or pumped on demand to provide a fluid flow as needed or may be under constant pressure and valved for quick flow. Valves, pressure sources, and/or pumps can be in communication with a processor operable to control fluid flow in the various channels in response to feedback from various sensors including any optical or electrical detection sensors in the sensing region 103.

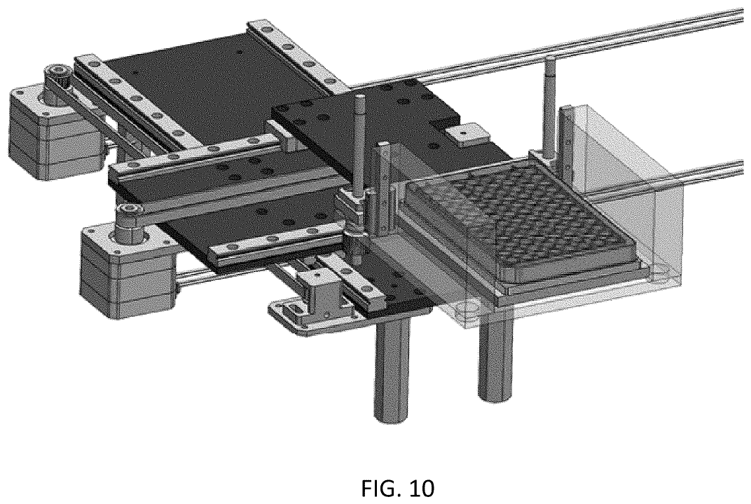

Application of the trigger flow from the trigger channel 113 into the sorting region 105 operates to divert the sample fluid flow from the sample flow channel 107 into a branch channel 115 and, when timed based on measured or calculated flow rates and distance between the sensing region 103 and sorting region 105, can be controlled by the processor to divert only a target particle after detection in the sensing region 103. The trigger channel 113 may be preferably aligned with the branch channel 115 such that the leading edges of their respective outlet and inlet are substantially aligned with each other on opposite sides of the sample flow channel 107. The branch channel 115 is preferably at an acute angle 123 relative to the direction of sample flow in the sample flow channel 107. As such, diversion into the branch channel 115 requires less force from the trigger flow as the change of direction for the particle is less severe than if the branch channel 115 intersected with the sample flow channel 107 at a right angle or obtuse angle. The trigger channel 113 may be substantially perpendicular to the direction of sample flow in the sample flow channel 107 at the sorting region 105. The target particle, once diverted to the branch channel 115, may then be directed into a target channel 111 for further handling. The target channel may lead to a collection chamber or onboard processing or analysis (e.g., cell lysis or additional processing steps or assays such as amplification and/or sequencing). In preferred embodiments, the target channel 111 leads to a nozzle 119 from which individual droplets containing single target particles can be directed into a well (e.g., in a 96 well plate) by XY axis manipulation of the nozzle 119, cartridge, and/or the well. The well(s) and/or the cartridge can be placed on a multi-axis stage controllable by the processor to time and locate target particle droplet dispensing over a desired well. An exemplary XY stage for manipulating plate of wells is shown in FIG. 10. In some embodiments, a 3-dimensional stage may be provided to allow for changes in vertical positioning of the nozzle relative to the wells (i.e., changing the distance the droplet falls from the nozzle to the well).

Returning to the exemplary microfluidic chip depicted in FIG. 1, the branch channel 115 may lead to an intersection of the target channel 111 and a carrier channel 117 providing carrier fluid to the circuit. Carrier fluid, as with the trigger fluid, may be provided from an onboard reservoir or carrier fluid source located in the instrument through an inlet port and interface. The carrier channel 117 can be pressurized with carrier fluid with just enough force to resist intrusion of sample fluid into the branch channel 115 under normal operating conditions. In preferred embodiments, the amount of pressure or force should be limited to a level that does not block or prevent flow from the sample flow channel into the waste channel. Upon detection of a target particle and application of a trigger flow, the additional pressure in the sample flow channel 107 at the sorting region 105 may then be enough to overcome the resistive pressure of the carrier fluid such that the sample flow including the target particle is directed into the branch channel 115 and target channel 111 toward the nozzle 119 for dispensing. The nozzle 119 or other processing element may provide enough resistance to substantially prevent flow in the target channel 111 and carrier channel 117 during normal operation and until application of a trigger flow from the trigger channel 113. In some embodiments, carrier fluid may continuously flow in the carrier channel 117 during normal operation. The carrier fluid may optionally flow into a carrier waste channel 127 during normal operation. In preferred embodiments no carrier waste channel 127 is provided, however. In such embodiments, any carrier fluid flow during normal operation (e.g., without application of a trigger flow) will be directed through the target channel 111 into the nozzle 119 and potentially the waste channel 109 as well. A waste well may be designated for any droplets of carrier fluid that may be dispensed during normal operation that would be lacking a target particle. In some embodiments, the processor may maintain the nozzle 119 over a target well and direct the nozzle 119 over such a waste well(s) when a droplet is anticipated in which a target particle is not contained. In other embodiments, the processor may direct the nozzle 119 over such a waste well(s) during normal operation until a target particle is detected and diverted such that any inadvertent drops would fall into the waste well(s).

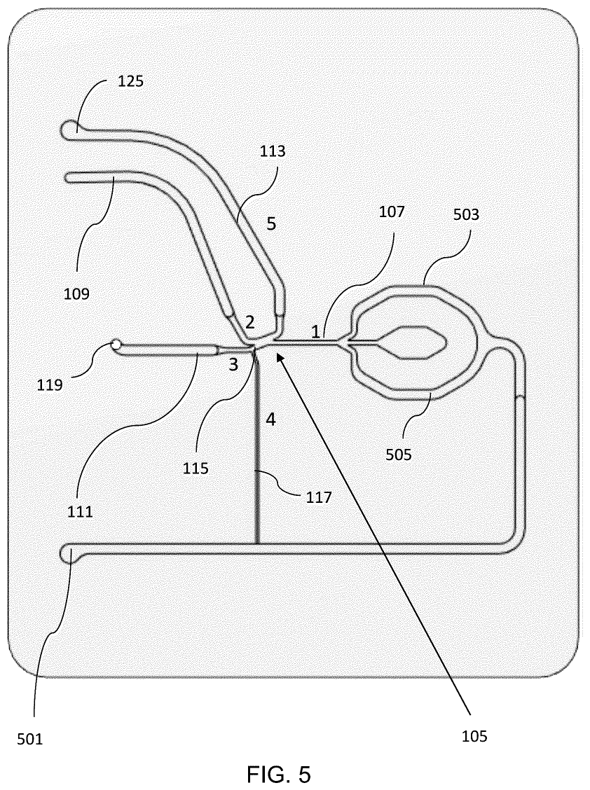

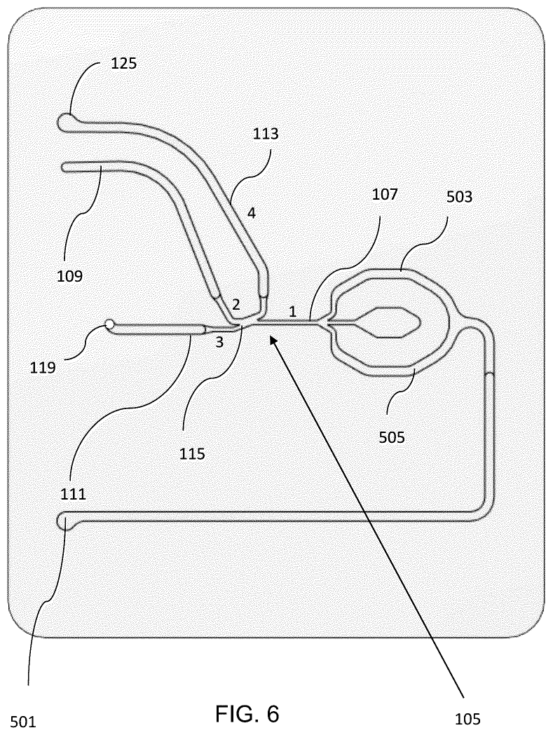

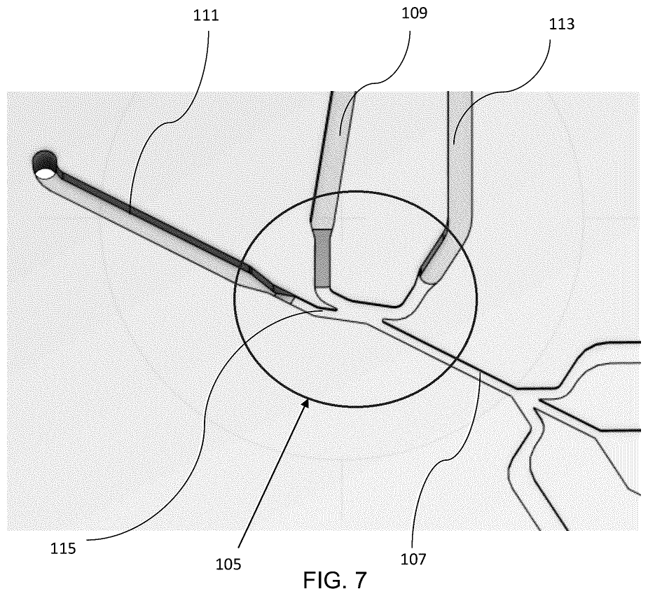

In certain embodiments, a sheathing or focusing fluid may be provided through opposing first 503 and second 505 sheath channels as shown in FIGS. 5 and 6. The sheathing fluid may be introduced on either side of the sample flow channel 107 upstream of the sorting region 105 or, in certain embodiments, upstream of the sensing region. The two sheath channels 503, 505 may be fed by a single fluid source which, as with the carrier and trigger fluids discussed above, may be provided by an onboard reservoir or an off-chip fluid source via an interface and inlet 501. In certain embodiments, the sheath fluid and carrier fluid may be the same and may be provided by the same source where, for example, the carrier channel 117 branches off of a common feed upstream of the two sheath channels 503, 505 as shown in FIG. 5. FIGS. 5 and 6 further illustrate embodiments in which no carrier waste channel is used. In certain embodiments, as depicted in FIG. 6, focusing of the sample flow in the center of the sample flow channel 107 using the sheath channels 503, 505 may obviate the need for a carrier channel altogether. In some embodiments, an arrangement similar to FIG. 6 may be used but without the sheath channels 503, 505 such that no carrier or sheath fluid is present and only sample and trigger channels flow into the sorting region.

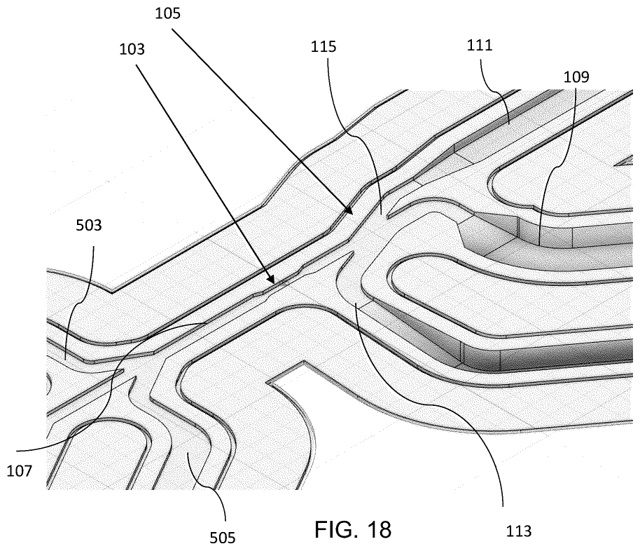

Providing predictable flow characteristics can be paramount to accurate particle diversion and sorting in the sorting region. Accordingly, limiting flow calculations to two dimensions and avoiding three-dimensional flow may be preferable. In order to avoid three-dimensional flow, channels depths may be limited at least in the sorting region 105. However, limited channel depth may not be necessary or desirable outside of that region. Accordingly, as shown in FIG. 7, one or more of the sample flow 107, trigger 113, branch 115, target 111, waste 109, and carrier (not shown) channels may have a shallow depth in the sorting region 105 and/or sensing region and a greater depth outside of those areas with a ramped or stepped transition as they approach or depart the sensing or sorting region 105. In various embodiments, the shallow depth may be about 100 μm or less, 150 μm or less, about 200 μm or less, about 250 μm or less, about 300 μm or less, about 350 μm or less, about 400 μm or less, about 500 μm or less, about 600 μm or less, or about 700 μm or less, with one or more of the channels increasing to correspondingly greater depths outside of the sorting region. In certain embodiments, three-dimensional flow characteristics can be avoided in channels of greater depths by limiting the pressure of the sample flowing in the sample flow channel 107. In certain embodiments, the shallow depth may be 200 μm or less in the sorting region 105, and one or more of the channels may have a depth greater than 200 μm outside of the sorting region 105. In some embodiments all of the channels may have a depth of 100 μm or less in the sorting region 105. FIG. 18 illustrates an additional embodiment of a sensing 103 and sorting region 115 having reduced channel depths within the regions relative to the channel depths outside of those regions along with ramped transitions between depths. As shown, in certain embodiments one or more of the channels may also have increased channel widths outside of the sensing 103 and/or sorting region 115 and the ramped transitions may include ramped or stepped narrowing or widening of the channel(s).

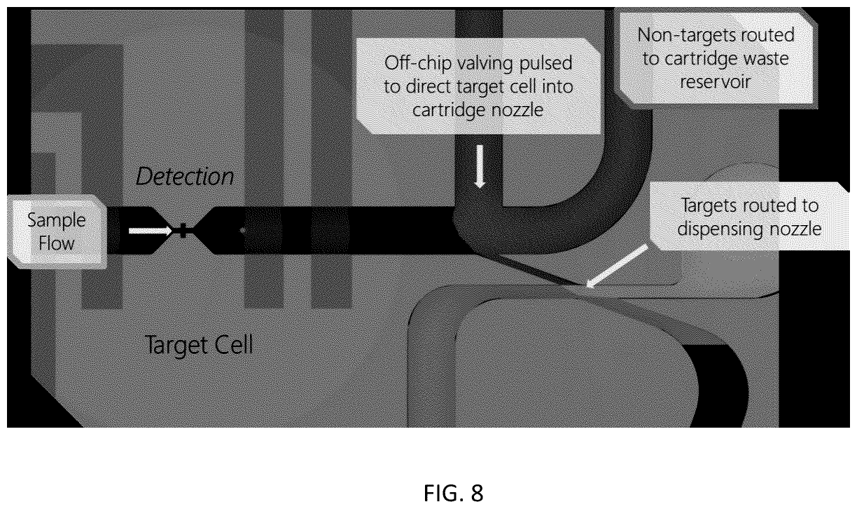

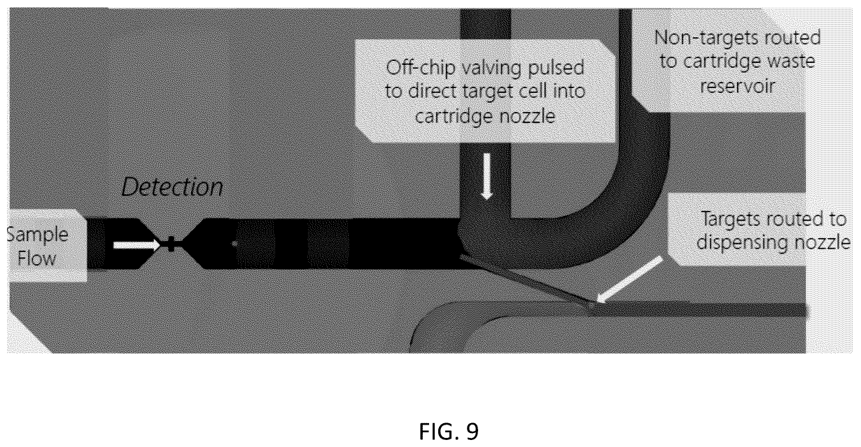

Exemplary particle detection and diversion processes are illustrated in FIGS. 8 and 9. FIG. 8 illustrates a process including an optional carrier waste channel while FIG. 9 shows a process without the carrier waste channel. In either instance, as discussed above, the sample flow passes through a sensing region or detection zone and, upon detection of a target particle, a trigger flow is applied from a trigger channel by pulsing trigger fluid using off-chip valving. The trigger fluid diverts the sample flow including the target particle into a branch channel, overcoming pressure or flow of carrier fluid in the carrier and target channels to direct the target particle through the branch channel and into the target channel for dispensing via a nozzle. In normal flow (e.g., when no target particle has been detected) the sample flow including any non-target particles is routed to a waste channel into a waste reservoir.

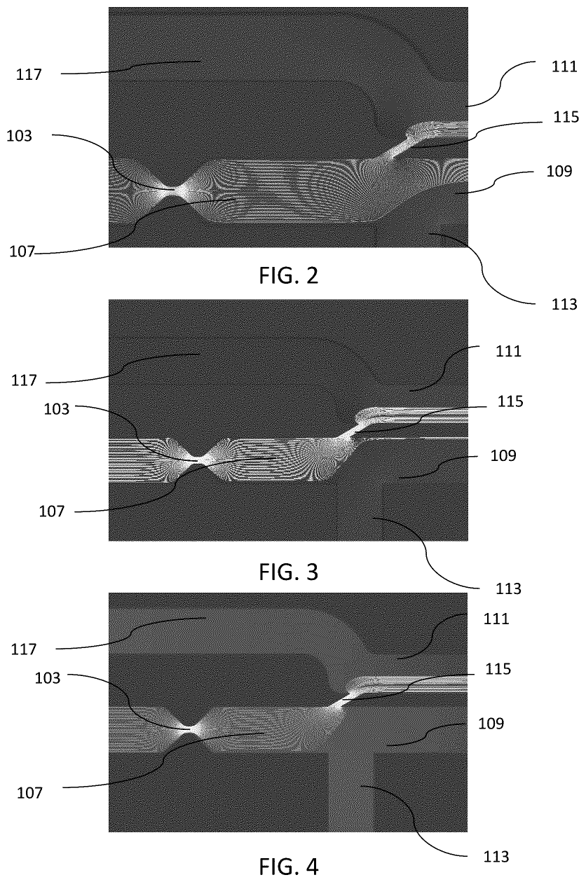

The diversion process at various trigger fluid pressures/flow rates is illustrated in FIGS. 2-4 where the lighter lines indicate sample fluid flow. In FIG. 2 an insufficient trigger flow is applied allowing much of the sample fluid to continue along the sample flow channel 107 into the waste channel 109 while some of the sample fluid is directed through the branch channel 115 to the target channel 111. Additional trigger flow pressure is applied in FIG. 3, but it is still insufficient for complete switching/diversion of sample fluid into the branch channel 115. The situations depicted in FIGS. 2 and 3 are undesirable because diversion of the target particle would be unpredictable and target particles may be lost into the waste channel. In FIG. 4, sufficient trigger flow has been applied to provide complete switching such that all of the sample flow is diverted into the branch channel 115 ensuring that any target particles therein are directed to the target channel 111. While diversion/switching control is discussed herein with respect to trigger fluid pressure, those of skill in the art will understand that pressure differential among the trigger fluid, carrier fluid, and sample fluid (and sheath fluid where applicable) can dictate flow characteristics in the chip. Accordingly, flow switching may be altered not only by altering trigger flow pressure but also by altering pressure of the sample flow and/or carrier flow in order to tune particle diversion.

Particle sensing or detection may be via any known method capable of providing a machine-readable signal indicating presence of a target particle. As noted, the instrument may include a processor coupled to a tangible, non-transient memory storing instructions for executing the various methods discussed herein. The processor can receive data from the sensor as a particle passes through the sensing region and, using predictive algorithms, time application of the trigger fluid downstream to divert the particle if it is identified as a target particle. The processor can further track the status of distribution wells or other receiving containers via feedback and control of, for example, the 2-dimensional stage discussed above and can position the well and nozzle to control target particle distribution.

As noted above, in certain embodiments the branch channel may be arranged at an acute angle to the direction of sample flow in the sample flow channel. This can allow for easier diversion of target particles as the change in direction from sample flow to branch channel is less severe. Accordingly, the trigger flow required to achieve that diversion may be less, thereby avoiding high pressures which can complicate flow modelling. In some embodiments, the trigger channel may be positioned such that it is roughly parallel to the branch channel. For example, the angle of the branch channel with respect to the flow of sample fluid is the sample flow channel may be about 22 degrees while the trigger channel joins the sample flow channel on the opposite side thereof at an angle of about 22 degrees. In certain embodiments, the trigger channel may join the sample flow channel at approximately a right angle, thereby maximizing the lateral force imposed by the trigger flow in diverting target particles into the opposite branch channel inlet.

In some embodiments, the channels leading into and/or away from the sample flow channel at the sorting region may widen or narrow as the approach the sorting region. Accordingly, angles between the sample flow channel and either side of the trigger and/or branch channels may not add to 180 degrees. For example, where the acute angle between the branch channel and the sample flow channel is about 22 degrees, the corresponding obtuse angle between the opposite side of the branch channel and the sample flow channel may be 160 degrees or more (indicating a narrowing branch channel leading away from the sorting region.

In various embodiments, one or more of the fluids (e.g., sample fluid, carrier fluid, sheath fluid, and trigger fluid) may by immiscible with one or more of the other fluids. For example, a sample fluid may be aqueous while the sheath fluid, trigger fluid, and/or carrier fluid may be an oil. In some embodiments, the flow achieved may be somewhat laminar such that the fluids do not mix even if not immiscible with one another. In some embodiments, all fluids may be aqueous (e.g., PBS buffer) including sample, carrier, and/or trigger.

### Cartridge Architecture

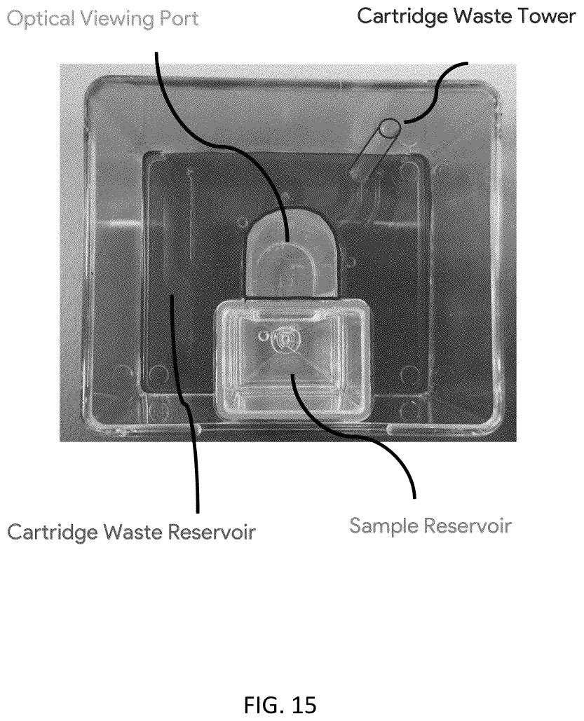

FIG. 15 depicts an exemplary particle sorting cartridge of the invention wherein the microfluidic chip and channels described above may be positioned at the bottom of the cartridge with fluidic reservoirs positioned above. As shown in FIG. 15, a sample reservoir is included into which a user would load the sample to be analyzed. In preferred embodiments, loading the sample into the sample reservoir and loading the cartridge into the instrument may be the only physical steps required from a user before the instrument can sense and isolate target particles from the sample. Any remaining required fluids may be stored onboard the instrument and introduced to the respective channel via an interface with the cartridge as discussed above. In some embodiments, these fluids may be loaded or stored in additional reservoirs in the cartridge. As illustrated in FIG. 15, the sample reservoir may have an angled floor (e.g., funnel shaped or otherwise slanted toward a single lowest point). The inlet to the sample flow channel may be placed at the lowest point in the angled floor to prevent settling of particles and maximize sample recovery into the chip. The instrument may have a stirring bar or other agitating mechanisms located in the sample reservoir to mix the sample, distribute particles for better sorting, and/or prevent settling. As discussed above, the inlet to the sample flow channel may include a filter which may comprise a series of microposts. These features may be injection molded directly within the cartridges and allow for consistent performance of the fluidics. Larger aggregates that cause potential clogging can be caught upstream of the detection or sensing region and therefore do not cause runs to fail. Further, such filters, especially the micropost layout depicted, can improve the single cell suspension nature of samples before they enter the detection region by breaking up and dispersing any groups of cells or other particles as they enter the channel.

Also as shown in FIG. 15, a waste reservoir may be positioned in the cartridge above the microfluidic chip. The waste reservoir can form a well into which waste, including any non-target particles and sample, may flow through the waste channel as described above. If the waste channel emptied directly into the floor of the waste reservoir above it, accumulated waste would continue to increase the backpressure on the waste channel and thereby complicate flow calculations and lead to inconsistent flow and/or pressures in the chip. In order to avoid such issues, in certain embodiments, the waste channel may empty into the waste reservoir via one or more towers having a limited volume and an outlet toward the top of the reservoir. Accordingly, waste will quickly fill the tower and then spill out and down into the waste reservoir. The backpressure will therefore be limited by the volume of the tower(s) and will not continue to increase as the reservoir fills.

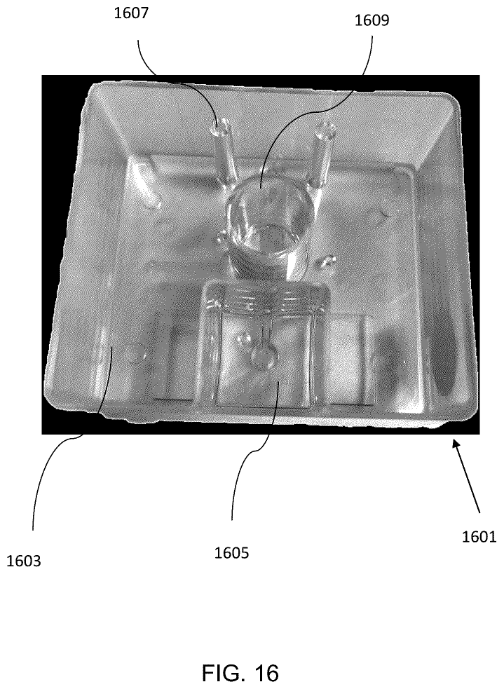

FIG. 16 illustrates another exemplary cartridge 1601 from above having a sample reservoir 1605, a larger waste reservoir 1603, and two towers 1607 through which waste enters the waste reservoir from the waste channel. Additionally, a viewport 1609 may be included where a sensor, for example for forward scattering or axial light loss occlusion, or an illumination or excitation source in the instrument is positioned above the cartridge 1601 in order to provide an unobstructed optical path through the reservoir(s) to the sensing region below for target detection. Otherwise, fluids in the reservoir(s) could interfere with light used for excitation or detection or other sensor functions. As shown in FIG. 16, the viewport can include at least one wall (e.g., a cube or a cylinder as depicted) operable to hold back fluid in the reservoir above the sensing region in the chip below.

In certain embodiments, sample reservoirs may have a volume between about 3 mL and about 5 mL. Waste reservoirs may have a volume of about 10 mL, about 20 mL, about 30 mL, about 40 mL, about 45 mL, about 60 mL about 75 mL, about 100 mL, or more. Towers in the waste reservoir may have openings at least about 5 mm, about 10 mm, about 15 mm, about 20 mm, about 25 mm, or more above the floor of the waste reservoir. The towers may have a volume of about 0.035 mL in certain embodiments. Tower height and volume can be calculated and manipulated to achieve a desired amount of back pressure in the system.

In various embodiments, cartridges may be constructed of bioinert materials to preserve cell viability. The cartridge material may have low autofluorescence background to avoid interference with laser or photodetector-based detection approaches. Cartridges may have transparent materials in certain locations, for example at the viewport, in order to allow forward scatter/axial light loss measurements. Exemplary cartridge materials may include injection molded plastics such as a cyclic olefin polymer or a cyclic olefin copolymer or, for example, PMMA, polystyrene, or polycarbonate.

### Device-Cartridge Interface

As discussed above, the device may interact with the cartridge by controlling fluidic movement within the channels (e.g., driving sample, trigger, and/or carrier flow), interacting with the sensing region to detect target particles, droplet sensing at the nozzle, and positioning the nozzle relative to target wells for dispensing. Many of these interactions require precise alignment of the cartridge in the instrument including mechanical alignment, pneumatic interfaces, optical or other sensors, and/or electrical connections. In particular, detection elements such as optical sensors may be present in the instrument but must be precisely aligned with the sensing region and, where applicable, viewport in the cartridge in order to identify target particles such as single cells in the microfluidic sample flow channel. Additionally, pressure manifolds for driving fluid flow from cartridge-based reservoirs and port interfaces for introducing instrument-based fluids into the microfluidic chip must be properly aligned to function. Furthermore, the stage and nozzle should be aligned to ensure accurate dispensing into target wells.

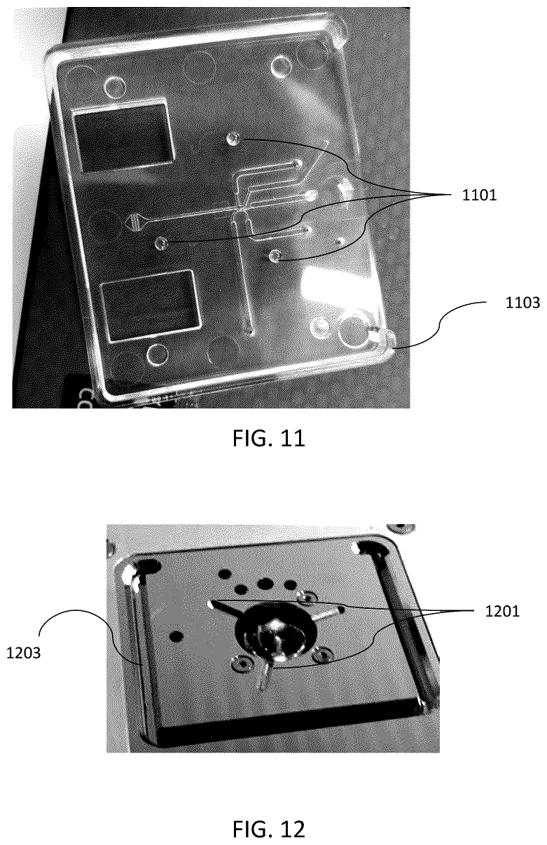

While traditionally, alignment may require complicated user input along with motorized or manual positioning and optical testing/verification, systems and methods of the invention may include alignment features present on the cartridge and/or corresponding cradle in the instrument to avoid the need for such positioning systems. Such features may include interacting raised or recessed elements configured to interact to precisely position the cartridge in a cradle in the instrument. Features may be asymmetric to only allow the cartridge to be placed in a single orientation. For example, the cartridge may comprise a skirt while the instrument cradle comprises a complimentary valley to receive the skirt. The skirt and valley may only partially extend around the perimeter of the cartridge (e.g., three sides of a square) such that a single orientation is permitted. The skirt and valley can also provide a rough alignment depending on the relative sizes of the two elements. In certain embodiments, a skirt and valley may be configured to provide a rough alignment while additional features provide final alignment. For example, kinematic coupling may be used. In certain embodiments one, two, or preferably three hemispherical features may be used on the mating surface of one of the instrument cradle or cartridge with corresponding v-grooves on the other mating surface to precisely align the cartridge in the cradle. FIG. 11 illustrates exemplary alignment features on the base of a cartridge. As shown, a skirt 1103 extends around three sides of the cartridge and matches a groove 1203 in the cradle on the device providing rough alignment and ensuring one-way fit. Additional, fine alignment can be provided by the aforementioned kinematic coupling including hemispherical or ball shaped features 1101 on the cartridge or instrument that fit in corresponding grooves 1201 in the instrument to provide fine alignment.

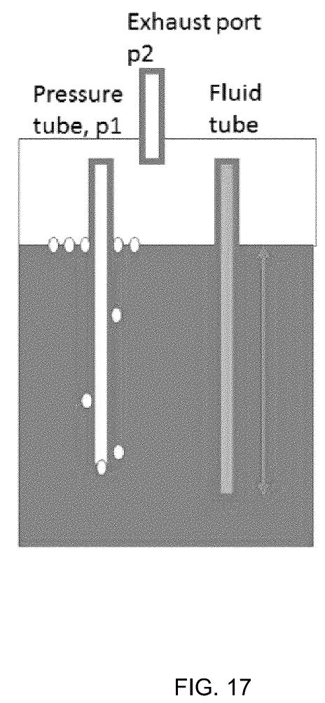

As noted, alignment can be used to properly couple fluid inlets to cartridge channels to fluid sources on the instrument. Alignment may also be used to position and seal cartridge reservoirs to pneumatic manifolds, plungers or other devices for manipulation of fluids on the cartridge. For example, the sample reservoir can be sealed against a pneumatic interface or manifold through which air pressure can be applied to the sample fluid to drive it into the sample flow channel inlet and on through the microfluidic chip. In certain embodiments, the pressure manifold may connect to liquid reservoirs that contain trigger, carrier, sheath, or sample fluids. These reservoirs can include inlet tubing, outlet tubing that never contacts the liquid that vents, and outlet tubing that contacts and can be submerged into the liquid to allow for fluid flow out. Air or liquid contacting valves can be used to control and adjust pressures in a reservoir. When using the layout shown in FIG. 17 for a reservoir, pressure sensing in the different lines can be used to determine and track fluid levels in the reservoir. In some embodiments, a low fluid level threshold can trigger the device to provide audible and/or visual alerts to a user to refill the reservoir and/or may automatically pause sample flow to ensure no loss of target particles. In certain embodiments, syringe pumps can be used to provide fluid flow.

Furthermore, the various fluids (e.g., sample, trigger, carrier, and/or sheath) can be independently driven or valved downstream of a pressure-controlled reservoir such that flow in each channel can be manipulated and toggled individually. Accordingly, in certain embodiments, upon detection of a target particle for example, sample flow (and sheath fluid flow where applicable) may be paused through the sensing region while the device continues to drive the target particle through the branch and target channels using the trigger and/or carrier fluids. The processor, in operable communication with each fluidic system (e.g., pressure manifold) and the various sensors can determine when the target particle should be routed to the nozzle and operate as normal by applying a trigger fluid but, instead of continuing to drive sample flow, can reduce or entirely pause pressure to the sample reservoir. This can accelerate dispensing time of the target particle by allowing for trigger fluid to flow into the cartridge without unnecessary additional sample flowing to the nozzle or increasing the driving pressure of the trigger fluid and can also serve to pause target detection while the current particle is sorted by stopping sample from being processed or wasted. Accordingly, target particles occurring in the sample in rapid succession can still be accurately detected and sorted, ensuring maximum recovery from the sample. Valving of the fluid lines may be done without contacting the fluid (e.g. non-liquid-contacting valves to vent or continue pressurizing the reservoirs). In various embodiments, pressures used to drive sample flow and/or flow of trigger, carrier, sheath, or other fluids may be between about 0.01 PSI and about 10 PSI. Those of ordinary skill in the art will understand that increasing the pressure used to drive a fluid will result in a higher flow rate. The pressure may be tuned and varied during operation to achieve a desired flow rate to ensure accurate detection and sorting of particles in the sample flow.

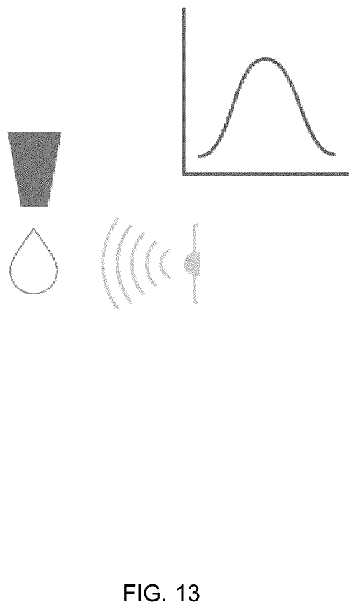

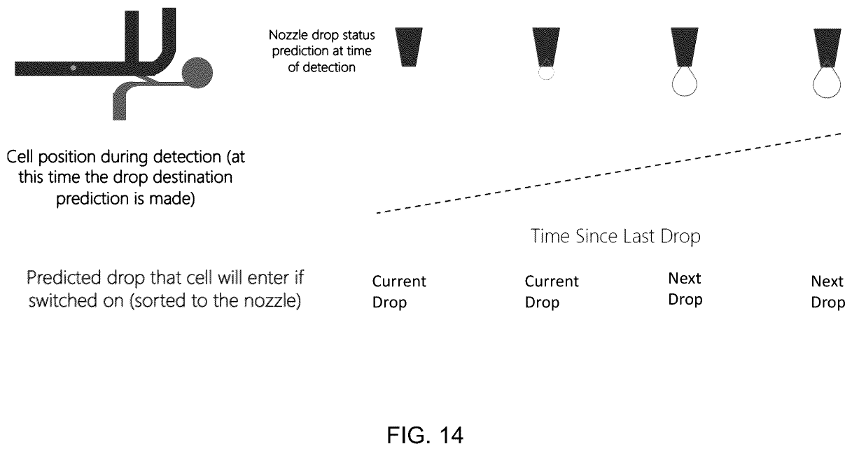

In various embodiments, the instrument may include one or more drop sensors operable to monitor meniscus formation and/or breakoff at the cartridge nozzle. An exemplary sensor is shown in FIG. 13 positioned relative to the nozzle. Drop sensors may include, for example, reflective optical sensors with transistor outputs, slot type optical sensors, acoustic sensors, camera elements, pressure sensors, capacitive sensors, and in-line flow sensors. The drop sensor can be in communication with the processor which can use information from the drop sensor to track target particle dispensing as shown in FIG. 14. For example, the drop sensor may provide a simple indication of when a drop falls which can be used to establish a drop frequency or rhythm to estimate when the next drop will fall. In some instances, the drop sensor can measure the size of the meniscus and/or the rate it is growing to more precisely estimate when a specific drop will fall from the nozzle. In either instance, the drop status at the time of particle detection can be used to estimate which drop the target particle will be present in. That information can be used by the system to position and track the placement of each target particle in the desired container or well for future analysis. Feedback from the drop sensor can also be used during initialization to adjust various pressures or flow rates in the system (carrier, trigger, sheath, or sample). The drop sensor signal may also be used to determine the size of the drop or the type of liquid.

Although various embodiments have been described as having particular features and/or combinations of components, other embodiments are possible having any combination or sub-combination of any features and/or components from any of the embodiments described herein. The specific configurations of the various components can also be varied. For example, the size and specific shape of the various components can be different than the embodiments shown, while still providing the functions as described herein. Thus, the breadth and scope of the invention should not be limited by any of the above-described embodiments. The previous description of the embodiments is provided to enable any person skilled in the art to make or use the invention. While the invention has been particularly shown and described with reference to embodiments thereof, it will be understood by those skilled in the art that various changes in form and details may be made therein without departing from the spirit and scope of the invention.

### Particle Sensing

As discussed above, particle sorting may occur after particle detection or analysis in a sensing region. Exemplary particle sensing or detection methods include optical, electrical, and fluorescence detection such as those described in US Pub. 2020/0096436 and U.S. Pat. Nos. 9,201,043 and 10,350,602, incorporated herein by reference in their entirety. In certain embodiments, optical detection methods such as forward scatter or axial light loss may be used. Both methods provide information regarding particle size and are well characterized for use in flow cytometry. Forward scatter measures light scattered by particles in directions substantially parallel to the excitation light source while axial light loss is the measurement of the total light lost from the laser beam along the propagation direction when a particle passes through the beam.

As discussed above, alignment of the cartridge with respect to optical sensor is extremely important to ensure accurate detection or analysis of the particles in the sensing region of the cartridge. As the optical components may be located on the instrument while the sample, target particles, and sensing region are all located on the cartridge, achieving this precise alignment can be challenging and introduces an opportunity for user error to impact the system. As discussed above, physical alignment tools such as kinematic coupling features may be used to reduce chances for error. By precisely manufacturing and providing a monolithic unit including both the optics module and the cartridge cradle (with alignment features), much of the alignment responsibility can be removed from the end user and accomplished with machine precision at the manufacturing stage. Such a combination may be achieved using an interference fit of components including the use of shrink fitting (e.g., between the cartridge cradle and an optics plate).

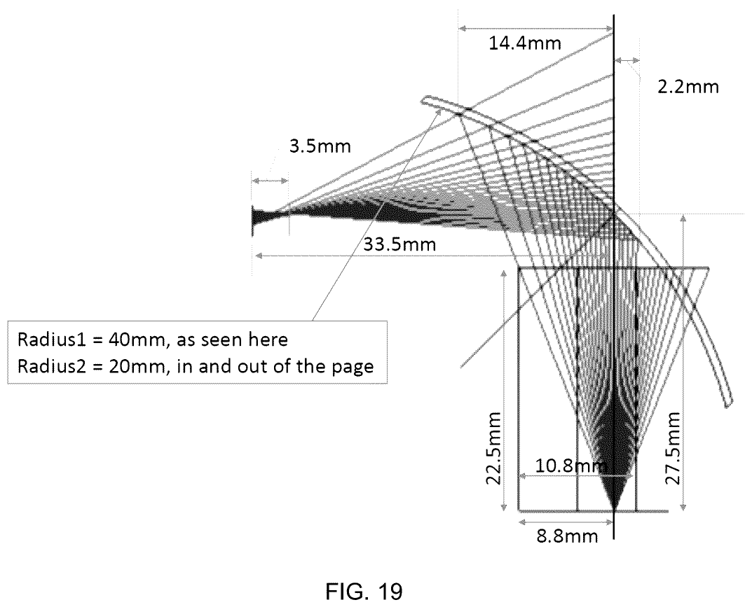

As discussed above, in many embodiments, optical elements may be positioned below and/or above the cartridge to direct light from a source to the sensing region and to receive light reflected or passed through the sensing region. In some embodiments, some of these optical elements may therefore be placed on a moving part (e.g., a hinged or otherwise moving lid allowing cartridge insertion and removal). When such optical elements are routinely moved relative to both the cartridge and other optical components on the instrument, another opportunity for alignment errors is introduced. In certain embodiments, optical detectors used in systems and methods of the invention may include toroidal or off-axis parabolic mirrors to compensate for minor misalignments such as those that might result from a lid opening and closing. The principle is illustrated in FIG. 19 showing how light arriving at a toroidal mirror from a variety of different angles can still be directed to the same point.

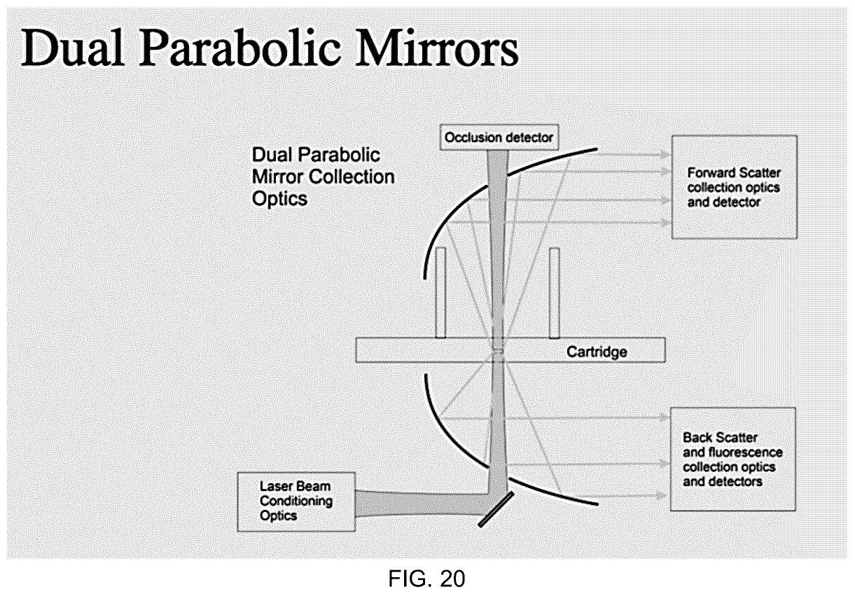

FIG. 20 shows an exemplary incorporation of off-axis parabolic mirrors into a combined forward scatter and occlusion detection system compatible with the cartridges and instruments described above. Such an arrangement allows for optimal optical detection of both traditional forward scatter as well as occlusion (axial light loss) capture on detectors even in a moving part such as a lid as discussed. As shown, parabolic mirrors positioned above and below the cartridge cradle (and cartridge therein) can redirect and/or focus light arriving at the mirrors from a variety of angles for forward scatter, back scatter, and/or fluorescence detection and analysis. The toroidal or parabolic mirrors may be dichroic in order to allow certain wavelengths of light from the source to pass therethrough or have holes that allow for light to pass through certain regions (e.g., to provide light for fluorescence excitation, scatter analysis, and/or occlusion detection).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A particle sorting cartridge, comprising:
a sorting region comprising:
a sample flow channel leading from a sensing region into a waste channel and having at least a first side and a second side substantially opposite the first side;
a branch channel having an inlet on the first side of the sample flow channel and positioned at an angle acute to a direction of flow in the sample flow channel at the sorting region; and
a trigger channel having an outlet on the second side of the sample flow channel,
wherein each of the sample flow channel, waste channel, branch channel, and trigger channel have a channel depth of about 5200 μm or less within the sorting region, and
wherein one or more of the sample flow channel, waste channel, branch channel, and trigger channel have a channel depth greater than about 5200 μm before or after the sorting region.

2. The particle sorting cartridge of claim 1, wherein branch channel leads away from the sorting region to a target channel.

3. The particle sorting cartridge of claim 2, wherein the target channel is operably associated with a nozzle.

4. The particle sorting cartridge of claim 2, further comprising a carrier channel connected to the target channel.

5. The particle sorting cartridge of claim 4, wherein the carrier channel and the branch channel intersect at an inlet to the target channel.

6. The particle sorting cartridge of claim 2, wherein each of the waste channel and trigger channel and one of the target channel or branch channel have a channel depth greater than about 5200 μm before or after the sorting region while the sample channel has a channel depth of 5200 μm or less between the sensing region and the sorting region.

7. The particle sorting cartridge of claim 1, wherein one or more of the sample flow channel, waste branch channel, and trigger channel having a channel depth greater than about 5100 μm before or after the sorting region comprise a ramped region wherein channel depth transitions from greater than about 5400 μm to about 5400 μm or less.

8. The particle sorting cartridge of claim 7, wherein the ramped region has an average slope between about 5 degrees and about 75-degrees.

9. The particle sorting cartridge of claim 1, wherein one or more of the sample flow channel, waste branch channel, and trigger channel having a channel depth greater than about 5400 μm before or after the sorting region comprise a stepped region wherein channel depth transitions from greater than about 5400 μm to about 5400 μm or less.

10. The particle sorting cartridge of claim 1, wherein the inlet has an upstream edge substantially aligned with an upstream edge of the outlet.

11. The particle sorting cartridge of claim 1, further comprising a first sheath channel having an outlet on the first side of the sample flow channel at a point upstream of the sorting region and a second sheath channel having an outlet on the second side of the sample flow channel at the point upstream of the sorting region.

12. The particle sorting cartridge of claim 11, wherein the first and second sheath channels intersect at an inlet fed by a single sheath fluid reservoir.

13. The particle sorting cartridge of claim 12, further comprising a carrier channel connected to the target channel, wherein the carrier channel is fed by the single sheath fluid reservoir.

14. A method for sorting a target particle from sample, the method comprising:
flowing a sample in a sample flow channel through a sensing region, through a sorting region, and into a waste channel, the sample flow channel having at least a first side and a second side substantially opposite the first side;
detecting a target particle in the sample at the sensing region and applying a trigger flow from a trigger channel having an outlet on the second side of the sample flow channel at the sorting region to divert the target particle from the sample flow channel into an inlet of a branch channel positioned on the first side of the sample flow channel at an angle acute to a direction of flow in the sample flow channel at the sorting region,
wherein each of the sample flow channel, waste channel, branch channel, and trigger channel have a channel depth of about 5200 μm or less within the sorting region, and
wherein one or more of the sample flow channel, waste channel, branch channel, and trigger channel have a channel depth greater than about 5200 μm before or after the sorting region.

15. The method of claim 14, further comprising flowing the target particle through the branch channel to a target channel.

16. The method of claim 15, wherein the target channel is operably associated with a nozzle, the method further comprising dispensing the target particle from the nozzle.

17. The method of claim 15, further comprising flowing a carrier fluid in a carrier channel connected to the target channel, the carrier channel and the branch channel intersecting at an inlet to the target channel.

18. The method of claim 15, further comprising maintaining a fluidic pressure in a carrier channel connected to the target channel and the branch channel to resist flow of the sample from the sample channel into the inlet of the branch channel in the absence of the trigger flow.

19. The method of claim 14, wherein each of the waste channel, branch channel, and trigger channel have a channel depth greater than about 5400 μm before or after the sorting region while the sample channel has as channel depth of 5400 μm or less between the sensing region and the sorting region.

20. The method of claim 14, wherein one or more of the sample flow channel, waste branch channel, and trigger channel having a channel depth greater than about 5400 μm before or after the sorting region comprise a ramped region wherein channel depth transitions from greater than about 5100 μm to about 5100 μm or less.

21. The method of claim 20, wherein the ramped region has an average slope between about 5 degrees and about 30 degrees.

22. The method of claim 14, wherein one or more of the sample flow channel, waste branch channel, and trigger channel having a channel depth greater than about 100 μm before or after the sorting region comprise a stepped region wherein channel depth transitions from greater than about 100 μm to about 100 μm or less.

23. The method of claim 14, wherein the inlet of the branch channel has an upstream edge substantially aligned with an upstream edge of the outlet of the target channel.

24. The method of claim 14, further comprising flowing a sheath fluid in a first sheath channel having an outlet on the first side of the sample flow channel at a point upstream of the sorting region and a second sheath channel having an outlet on the second side of the sample flow channel at the point upstream of the sorting region.

25. The method of claim 24, wherein the first and second sheath channels intersect at an inlet fed by a single sheath fluid reservoir.

26. The method of claim 25, further comprising flowing a carrier fluid in a carrier channel connected to the target channel, the carrier channel and the branch channel intersecting at an inlet to the target channel, wherein the carrier channel is fed by the single sheath fluid reservoir.

27. A particle sorting cartridge, comprising:
a microfluidic chip comprising:
a sample flow channel leading laterally from a sample reservoir positioned above the microfluidic chip, through a sensing region and into a sorting region; and
a waste channel leading laterally from the sorting region to a waste reservoir inlet;
a waste reservoir positioned above the microfluidic chip; and
a viewport positioned vertically within one or more of the waste reservoir and the sample reservoir and directly above the sensing region, the viewport comprising at least one vertical wall operable to provide an optical path through one or more of the waste reservoir and the sample reservoir to the sensing region from above, the optical path being free from sample fluid and waste fluid.

28. The particle sorting cartridge of claim 27, further comprising a tower positioned vertically in the waste reservoir above the waste reservoir inlet, the tower having an outlet spaced vertically above a floor of the waste reservoir, the tower configured such that waste fluid from the microfluidic chip flows laterally through the waste channel into the waste reservoir inlet then vertically through the tower, then out of the outlet into the waste reservoir below the outlet, thereby limiting backpressure in the waste channel as waste fluid accumulates in the waste reservoir.

29. The particle sorting cartridge of claim 28, wherein the waste reservoir has a volume of about 45 mL.

30. The particle sorting cartridge of claim 29, wherein the tower has a volume of about 0.035 mL.

31. The particle sorting cartridge of claim 28, wherein the outlet is positioned on the tower about 20 mm above the waste reservoir inlet.

32. The particle sorting cartridge of claim 27, wherein the waste reservoir has a greater volume than the sample reservoir.

33. The particle sorting cartridge of claim 27, wherein the sample reservoir comprises an angled floor operable to direct fluid in the sample reservoir to an inlet to the sample flow channel positioned at a lowest point in the sample reservoir.

34. The particle sorting cartridge of claim 27, wherein the sample flow channel comprises a filter between the inlet and the sensing region operable to allow target particles to pass therethrough but prevent passage of objects larger than a narrowest cross-sectional dimension in the sample flow channel without blocking flow through the filter.

35. The particle sorting cartridge of claim 34, wherein the filter comprises a plurality of microposts.

36. The particle sorting cartridge of claim 27, wherein the sample flow channel comprises a first side and a second side substantially opposite the first side, the microfluidic chip further comprising:
a branch channel having an inlet on the first side of the sample flow channel; and
a trigger channel having an outlet on the second side of the sample flow channel substantially aligned with the branch channel inlet.

37. The particle sorting cartridge of claim 36, further comprising a trigger channel inlet port operable to receive trigger fluid through a cartridge interface.

38. The particle sorting cartridge of claim 27, wherein the sample reservoir is operable to interface with a pressure manifold through a cartridge interface.

39. A method for sorting a target particle, the method comprising:
providing a particle sorting cartridge comprising:
a microfluidic chip comprising:
a sample flow channel leading laterally from a sample reservoir positioned above the microfluidic chip, through a sensing region and into a sorting region; and
a waste channel leading laterally from the sorting region to a waste reservoir inlet;
a waste reservoir positioned above the microfluidic chip; and
a viewport positioned vertically within one or more of the waste reservoir and the sample reservoir and directly above the sensing region, the viewport comprising at least one vertical wall operable to provide an optical path through one or more of the waste reservoir and the sample reservoir to the sensing region from above, the optical path being free from sample fluid and waste fluid;
flowing a sample fluid from the sample reservoir through the sample flow channel, sensing region, sorting region, and waste channel into the waste reservoir;
detecting a target particle through the viewport at the sensing region using an optical sensor; and
diverting the target particle into a branch channel at the sorting region.

40. The method of claim 39, wherein the particle sorting cartridge further comprises a tower positioned vertically in the waste reservoir above the waste reservoir inlet, the tower having an outlet spaced vertically above a floor of the waste reservoir, the method further comprising flowing waste fluid laterally through the waste channel into the waste reservoir inlet then vertically through the tower, then out of the outlet into the waste reservoir below the outlet, thereby limiting backpressure in the waste channel as waste fluid accumulates in the waste reservoir.

41. The method of claim 40, wherein the waste reservoir has a volume of about 45 mL.

42. The method of claim 41, wherein the tower has a volume of about 0.035 mL.

43. The method of claim 40, wherein the outlet is positioned on the tower about 20 mm above the waste reservoir inlet.

44. The method of claim 39, wherein the waste reservoir has a greater volume than the sample reservoir.

45. The method of claim 39, further comprising directing sample fluid in the sample reservoir to an inlet to the sample flow channel positioned at a lowest point of the sample reservoir using an angled floor of the sample reservoir.

46. The method of claim 39, further comprising filtering objects larger than a narrowest cross-sectional dimension in the sample flow channel from entering the sample flow channel using a filter between the inlet and the sensing region while allow target particles to pass therethrough without blocking flow through the filter.

47. The method of claim 46, wherein the filter comprises a plurality of microposts.

48. The method of claim 39, wherein the sample flow channel comprises a first side and a second side substantially opposite the first side, the microfluidic chip further comprising:
a branch channel having an inlet on the first side of the sample flow channel; and
a trigger channel having an outlet on the second side of the sample flow channel substantially aligned with the branch channel inlet, and
wherein diverting the target particle into the branch reservoir comprises applying a trigger flow from the trigger channel upon detection of the target particle.

49. The method of claim 48, wherein the particle sorting cartridge further comprises a trigger channel inlet port and applying the trigger flow comprises introducing fluid to the trigger channel via the trigger channel inlet port using a cartridge interface.

50. The method of claim 39, further comprising driving sample flow through the sample flow channel by applying pressure to the sample reservoir through a pressure manifold coupled to the sample reservoir through a cartridge interface.

51. A method for sorting a target particle from sample, the method comprising:
flowing a sample in a sample flow channel through a sensing region, through a sorting region, and into a waste channel, the sample flow channel having at least a first side and a second side substantially opposite the first side;
detecting a target particle in the sample at the sensing region;
applying a trigger flow from a trigger channel having an outlet on the second side of the sample flow channel at the sorting region to divert the target particle from the sample flow channel into an inlet of a branch channel positioned on the first side of the sample flow channel; and
stopping flow of the sample in the sample flow channel at some point during the application of the trigger flow.

52. The method of claim 51, wherein the branch channel inlet is positioned at an acute angle relative to flowing sample in the sample flow channel in the sensing region.

53. The method of claim 51, further comprising flowing the target particle through the branch channel to a target channel through application of the trigger flow.

54. The method of claim 53, wherein the target channel is operably associated with a nozzle, the method further comprising dispensing the target particle from the nozzle.

55. The method of claim 54, further comprising reinstituting flow of the sample in the sample flow channel after the target particle is dispensed.

56. The method of claim 53, further comprising reinstituting flow of the sample in the sample flow channel after the target particle enters the target channel.

57. The method of claim 53, further comprising reinstituting flow of the sample in the sample flow channel after the target particle is diverted to the branch channel.

58. The method of claim 51, further comprising flowing a carrier fluid in a carrier channel connected to the target channel, the carrier channel and the branch channel intersecting at an inlet to the target channel.

59. The method of claim 51, further comprising maintaining a fluidic pressure in a carrier channel connected to the target channel and the branch channel to resist flow of the sample from the sample channel into the inlet of the branch channel in the absence of the trigger flow.

60. The method of claim 51, wherein the inlet of the branch channel has an upstream edge substantially aligned with an upstream edge of the outlet of the target channel.

61. The method of claim 51, further comprising flowing a sheath fluid in a first sheath channel having an outlet on the first side of the sample flow channel at a point upstream of the sorting region and a second sheath channel having an outlet on the second side of the sample flow channel at the point upstream of the sorting region.

62. The method of claim 61, wherein the first and second sheath channels intersect at an inlet fed by a single sheath fluid reservoir.

63. A particle sorting system, comprising:
a particle sorting cartridge comprising:
a sensing region operable to detect a target particle in a sample;
a sorting region comprising:
a sample flow channel leading from the sensing region into a waste channel and having at least a first side and a second side substantially opposite the first side;
a branch channel having an inlet on the first side of the sample flow channel; and
a trigger channel having an outlet on the second side of the sample flow channel,
a sample fluid source operably associated with the sample flow channel;
a trigger fluid source operably associated with the trigger channel;
a processor operable to receive sensing data from the sensing region and, upon detection of a target particle, apply a trigger flow from the trigger fluid source and stop a sample flow from the sample fluid source at some point during the application of the trigger flow.

64. The particle sorting system of claim 63, wherein one or more of the sample fluid source and the trigger fluid source comprise a reservoir, a pressurized air supply, and valve controlled by the processor.

65. The particle sorting system of claim 63, wherein one or more of the sample fluid source and the trigger fluid source comprise a pump controlled by the processor.

66. The particle sorting system of claim 63, wherein the branch channel is positioned at an angle acute to a direction of flow in the sample flow channel at the sorting region.

67. The particle sorting system of claim 63, wherein the branch channel leads away from the sorting region to a target channel.

68. The particle sorting system of claim 67, wherein the target channel is operably associated with a nozzle.

69. The particle sorting system of claim 67, further comprising a carrier channel connected to the target channel.

70. The particle sorting system of claim 69, wherein the carrier channel and the branch channel intersect at a target channel inlet.

71. The particle sorting system of claim 63, wherein the inlet has an upstream edge substantially aligned with an upstream edge of the outlet.

72. The particle sorting system of claim 63, further comprising a first sheath channel having an outlet on the first side of the sample flow channel at a point upstream of the sorting region and a second sheath channel having an outlet on the second side of the sample flow channel at the point upstream of the sorting region.

73. The particle sorting system of claim 72, wherein the first and second sheath channels intersect at an inlet fed by a single sheath fluid reservoir.

74. The particle sorting system of claim 72, further comprising a sheath fluid source operably associated with the first and second sheath channels, the processor further operable to stop a sheath fluid flow upon detection of the target particle and application of the trigger flow.

75. A particle sorting cartridge, comprising:
a microfluidic chip comprising:
a sample flow channel leading laterally from a sample reservoir positioned above the microfluidic chip, through a sensing region and into a sorting region; and
a waste channel leading laterally from the sorting region to a waste reservoir inlet;
a waste reservoir positioned above the microfluidic chip; and
a tower positioned vertically in the waste reservoir above the waste reservoir inlet, the tower having an outlet spaced vertically above a floor of the waste reservoir, the tower configured such that waste fluid from the microfluidic chip flows laterally through the waste channel into the waste reservoir inlet then vertically through the tower, then out of the outlet into the waste reservoir below the outlet, thereby limiting backpressure in the waste channel as waste fluid accumulates in the waste reservoir.

76. The particle sorting cartridge of claim 75, wherein the waste reservoir has a volume of about 45 mL.

77. The particle sorting cartridge of claim 76, wherein the tower has a volume of about 0.035 mL.

78. The particle sorting cartridge of claim 77, wherein the outlet is positioned on the tower about 20 mm above the waste reservoir inlet.

79. The particle sorting cartridge of claim 75, wherein the waste reservoir has a greater volume than the sample reservoir.

80. The particle sorting cartridge of claim 75, wherein the sample reservoir comprises an angled floor operable to direct fluid in the sample reservoir to an inlet to the sample flow channel positioned at a lowest point in the sample reservoir.

81. The particle sorting cartridge of claim 75, wherein the sample flow channel comprises a filter between the inlet and the sensing region operable to allow target particles to pass therethrough but prevent passage of objects larger than a narrowest cross-sectional dimension in the sample flow channel without blocking flow through the filter.

82. The particle sorting cartridge of claim 81, wherein the filter comprises a plurality of microposts.

83. The particle sorting cartridge of claim 75, wherein the sample flow channel comprises a first side and a second side substantially opposite the first side, the microfluidic chip further comprising:
a branch channel having an inlet on the first side of the sample flow channel; and
a trigger channel having an outlet on the second side of the sample flow channel substantially aligned with the branch channel inlet.

84. The particle sorting cartridge of claim 83, further comprising a trigger channel inlet port operable to receive trigger fluid through a cartridge interface.

85. The particle sorting cartridge of claim 75, wherein the sample reservoir is operable to interface with a pressure manifold through a cartridge interface.

86. A method for sorting a target particle, the method comprising:
providing a particle sorting cartridge comprising:
a microfluidic chip comprising:
a sample flow channel leading laterally from a sample reservoir positioned above the microfluidic chip, through a sensing region and into a sorting region; and
a waste channel leading laterally from the sorting region to a waste reservoir inlet;
a waste reservoir positioned above the microfluidic chip;
a tower positioned vertically in the waste reservoir above the waste reservoir inlet, the tower having an outlet spaced vertically above a floor of the waste reservoir;
flowing a sample fluid from the sample reservoir through the sample flow channel, sensing region, sorting region, and then laterally through the waste channel into the waste reservoir inlet then vertically through the tower, then out of the outlet into the waste reservoir below the outlet, thereby limiting backpressure in the waste channel as waste fluid accumulates in the waste reservoir;
detecting a target particle through the viewport at the sensing region using an optical sensor; and
diverting the target particle into a branch channel at the sorting region.

87. The method of claim 86, wherein the waste reservoir has a volume of about 45 mL

88. The method of claim 87, wherein the tower has a volume of about 0.035 mL.

89. The method of claim 88, wherein the outlet is positioned on the tower about 20 mm above the waste reservoir inlet.

90. The method of claim 86, wherein the waste reservoir has a greater volume than the sample reservoir.

91. The method of claim 86, further comprising directing sample fluid in the sample reservoir to an inlet to the sample flow channel positioned at a lowest point of the sample reservoir using an angled floor of the sample reservoir.

92. The method of claim 86, further comprising filtering objects larger than a narrowest cross-sectional dimension in the sample flow channel from entering the sample flow channel using a filter between the inlet and the sensing region while allow target particles to pass therethrough without blocking flow through the filter.

93. The method of claim 92, wherein the filter comprises a plurality of microposts.

94. The method of claim 86, wherein the sample flow channel comprises a first side and a second side substantially opposite the first side, the microfluidic chip further comprising:
a branch channel having an inlet on the first side of the sample flow channel; and
a trigger channel having an outlet on the second side of the sample flow channel substantially aligned with the branch channel inlet, and
wherein diverting the target particle into the branch reservoir comprises applying a trigger flow from the trigger channel upon detection of the target particle.

95. The method of claim 94, wherein the particle sorting cartridge further comprises a trigger channel inlet port and applying the trigger flow comprises introducing fluid to the trigger channel via the trigger channel inlet port using a cartridge interface.

96. The method of claim 86, further comprising driving sample flow through the sample flow channel by applying pressure to the sample reservoir through a pressure manifold coupled to the sample reservoir through a cartridge interface.
